(19) United States
(12) Patent Application Publication
(10) Pub. No.: US 2023/0129956 A1
(43) Pub. Date: Apr. 27, 2023

(54) SURGICAL INSTRUMENTS AND METHODS INCORPORATING ULTRASONIC AND ELECTROSURGICAL FUNCTIONALITY

(71) Applicant: Covidien LP, Mansfield, MA (US)

(72) Inventors: Michael B. Lyons, Boulder, CO (US); Thomas E. Drochner, Longmont, CO (US); David J. Van Tol, Boulder, CO (US); Devon E. Scott-Drechsel, Superior, CO (US); James D. Allen, IV, Broomfield, CO (US)

(21) Appl. No.: 17/914,300

(22) PCT Filed: Mar. 11, 2021
(86) PCT No.: PCT/US2021/021902

§ 371 Date: Sep. 23, 2022

Related U.S. Application Data
(60) Provisional application No. 63/007,289, filed on Apr. 8, 2020.
(60) Provisional application No. 63/007,305, filed on Apr. 8, 2020.

(51) Int. Cl. A61B 18/14

(52) U.S. Cl. CPC A61B 18/1445; A61B 2018/00077; A61B 2018/1452

(57) ABSTRACT

A surgical instrument includes an ultrasonic transducer supported by a housing and an elongated assembly extending distally therefrom. The elongated assembly includes a jaw and a waveguide coupled to the transducer and defining a blade having upper and lower tissue-contacting surface and first and second lateral surfaces disposed therebetween that are coated with a material. The jaw is pivotable relative to the blade and includes a structural base having a backspan and first and second uprights extending from the backspan. The jaw further includes a jaw liner supported within the structural base and positioned to oppose the upper tissue-contacting surface with the first and second uprights disposed on either side of the blade. In an ultrasonic mode, ultrasonic energy produced by the transducer is transmitted along the waveguide to the blade. In an electrosurgical mode, electrosurgical energy is conducted between the blade and the first and second uprights.

### CROSS-REFERENCE TO RELATED APPLICATIONS

This application is a 371 National Stage Application of International Application No. PCT/US2021/021902, filed Mar. 11, 2021, which claims the benefit of U.S. Provisional Pat. Application Nos. 63/007,289 and 63/007,305, both filed on Apr. 8, 2020, the entire contents of each of which is hereby incorporated herein by reference.

### FIELD

The present disclosure relates to energy-based surgical instruments and, more particularly, to surgical instruments and methods incorporating ultrasonic and electrosurgical functionality to facilitate treating, e.g., sealing and/or dissecting tissue.

### BACKGROUND

Ultrasonic surgical instruments and systems utilize ultrasonic energy, i.e., ultrasonic vibrations, to treat tissue. More specifically, ultrasonic surgical instruments and systems utilize mechanical vibration energy transmitted at ultrasonic frequencies to coagulate, cauterize, fuse, seal, cut, desiccate, and/or fulgurate tissue to effect hemostasis. Ultrasonic surgical devices are used in many surgical procedures. An ultrasonic surgical device may include, for example, an ultrasonic blade and a clamp mechanism to enable clamping tissue against the blade. Ultrasonic energy transmitted to the blade causes the blade to vibrate at very high frequencies (e.g., 55,500 times per second), which allows for heating tissue to treat tissue clamped against or otherwise in contact with the blade.

Electrosurgical devices are also used in many surgical procedures. An electrosurgical device may include, for example, opposing jaw members operable to clamp tissue therebetween and conduct energy, e.g., RF energy, through clamped tissue to treat tissue.

Devices that combine ultrasonic and electrosurgical energy into a single multifunctional device and methods of using the same are generally known, but may not leverage the strengths of both technologies effectively. In particular, existing devices and methods of use thereof may not be optimized for the combined use of ultrasonic and electrosurgical energy.

### SUMMARY

As used herein, the term “distal” refers to the portion that is described which is further from a user, while the term “proximal” refers to the portion that is being described which is closer to a user. Further, any or all of the aspects described herein, to the extent consistent, may be used in conjunction with any or all of the other aspects described herein.

Provided in accordance with aspects of the present disclosure is a surgical instrument including a housing, an ultrasonic transducer supported by the housing, and an elongated assembly extending distally from the housing. The elongated assembly includes a waveguide formed from an electrically-conductive material and adapted to connect to a source of electrosurgical energy at a first potential. The waveguide is operably coupled to the ultrasonic transducer and includes a blade at a distal end portion thereof. The blade defines an upper tissue-contacting surface, a lower tissue-contacting surface, and first and second lateral surfaces disposed between the upper and lower tissue-contacting surfaces. The first and second lateral surfaces are coated with a material. The elongated assembly further includes a jaw pivotable relative to the blade between a spaced-apart position and an approximated position for grasping tissue between the blade and the jaw. The jaw includes a structural base formed from an electrically-conductive material and adapted to connect to a source of electrosurgical energy at a second potential different from the first potential. The structural base includes a backspan and first and second uprights extending from the backspan in spaced-apart relation to define a cavity therebetween. A jaw liner is supported within the cavity of the structural base and positioned to oppose the upper tissue-contacting surface of the blade in the approximated position with the first and second uprights disposed on either side of the blade.

In an ultrasonic mode of operation, ultrasonic energy is produced by the ultrasonic transducer and transmitted along the waveguide to the blade for treating tissue in contact with the blade.

In an electrosurgical mode of operation, electrosurgical energy is conducted between the blade and the first and second uprights to treat tissue disposed therebetween.

In an aspect of the present disclosure, each of the first and second uprights defines a tissue-contacting surface at the free end thereof. In such aspects, the tissue-contacting surfaces of the first and second uprights may be disposed in substantially parallel orientation relative to a tissue-contacting surface of the jaw liner. Additionally, the tissue-contacting surfaces of the first and second uprights may be angled inwardly towards one another and at an angle relative to a tissue-contacting surface of the jaw liner.

In another aspect of the present disclosure, each of the first and second uprights defines an inwardly-facing tissue-contacting surface. In such aspects, the inwardly-facing tissue-contacting surfaces may be disposed in substantially perpendicular orientation relative to a tissue-contacting surface of the jaw liner. Additionally or alternatively, in the approximated position of the jaw, the first and second lateral surfaces of the blade at least partially overlap with the tissue-contacting surfaces of the first and second uprights.

In still another aspect of the present disclosure, the lateral surfaces of the blade are disposed in substantially parallel orientation relative to one another. In such aspects, the lateral surfaces of the blade may be disposed in substantially parallel orientation with tissue-contacting surfaces of the first and second uprights.

In yet another aspect of the present disclosure, the upper tissue-contacting surface of the blade includes first and second surfaces meeting at an apex. In such aspects, the first and second surfaces of the upper-tissue contacting surface of the blade may be substantially parallel with respective angled tissue-contacting surfaces of the first and second uprights.

In still yet another aspect of the present disclosure, the material is an electrically-insulative material, e.g., Teflon® or polyphenylene oxide (PPO).

In another aspect of the present disclosure, inwardly-tapered surfaces extend from the first and second lateral surfaces of the blade at a distal end portion of the blade. In such aspects, the inwardly-tapered surfaces may be coated with an electrically-insulative material.

In yet another aspect of the present disclosure, the waveguide includes a body and the blade extending distally from the body. In such aspects, the body may be generally cylindrical with tapered surfaces defined between the generally cylindrical body and the first and second tissue-contacting surfaces of the blade. Additionally or alternatively, tapered surfaces are defined between the generally cylindrical body and the first and second lateral surfaces of the blade.

In still another aspect of the present disclosure, the surgical instrument further includes a plug assembly including an ultrasonic plug adapted to connect to an ultrasonic plug port of a surgical generator and an electrosurgical plug adapted to connect to an electrosurgical plug port of a surgical generator.

In another aspect of the present disclosure, the surgical instrument further includes at least one activation switch supported by the housing and configured to selectively initiate at least one of the ultrasonic mode of operation or the electrosurgical mode of operation.

In another aspect of the present disclosure, the jaw liner is formed from a compliant material.

Also provided in accordance with the present disclosure is a computer-implemented method of operating a surgical instrument capable of delivering both ultrasonic and RF energy. The method includes determining if a first switch is activated and, if so, determining whether or not tissue is grasped by an end effector of a surgical instrument. In a case where it is determined that tissue is grasped by the end effector, the method includes supplying both ultrasonic and RF energy to the end effector. In a case where it is determined that tissue is not grasped by the end effector, the method includes supplying only ultrasonic energy to the end effector.

In an aspect of the present disclosure, during the supply of both ultrasonic and RF energy to the end effector, the method may further include determining if a seal cycle for sealing tissue grasped by the end effector is complete and, if so, stopping the supply of RF energy to the end effector.

In another aspect of the present disclosure, in a case where it is determined that the seal cycle is complete, the method further includes changing a power level of the ultrasonic energy.

In yet another aspect of the present disclosure, the method further includes determining if tissue is dissected and, if so, stopping the supply of ultrasonic energy to the end effector.

In still another aspect of the present disclosure, in a case where the first switch is activated and it is determined that tissue is grasped by the end effector, the ultrasonic energy is supplied to the end effector at a relatively low power level and, in a case where the first switch is activated and it is determined that tissue is not grasped by the end effector, the ultrasonic energy is supplied to the end effector at a relatively high power level.

In still yet another aspect of the present disclosure, in a case where the first switch is not activated, it is determined whether a second switch is activated and, if so, the method includes supplying the ultrasonic energy to the end effector at the relatively high power level.

In another aspect of the present disclosure, in a case where the second switch is activated, the method further includes determining whether or not a large tissue load is detected and, if so, supplying, in addition to the ultrasonic energy, RF energy to the end effector.

Further provided in accordance with the present disclosure is a computer-implemented method of operating a surgical instrument capable of delivering both ultrasonic and RF energy. The method includes determining if a first switch is activated and, if so, determining whether a jaw of an end effector of the surgical instrument is open or closed. If the jaw is closed, the method includes determining whether tissue is grasped between the jaw and a blade of the end effector and, if so, supplying both ultrasonic and RF energy to the end effector. In a case where the switch is activated and the jaw is open, or where the switch is activated, the jaw is closed, and tissue is not grasped, the method further includes supplying only ultrasonic energy to the end effector.

In an aspect of the present disclosure, supplying only ultrasonic energy includes supplying the ultrasonic energy at a first power level (e.g., a higher power level) and wherein supplying both ultrasonic and RF energy to the end effector includes supplying the ultrasonic energy at a second, different power level (e.g., a lower power level).

### BRIEF DESCRIPTION OF THE DRAWINGS

The above and other aspects and features of the present disclosure will become more apparent in light of the following detailed description when taken in conjunction with the accompanying drawings wherein like reference numerals identify similar or identical elements.

FIG. 1 is a side, perspective view of a surgical system provided in accordance with the present disclosure;

FIG. 2A is an enlarged, side, perspective view of a distal portion of an end effector assembly of a surgical instrument of the surgical system of FIG. 1;

FIG. 2B is a transverse, cross-sectional view of the end effector assembly of FIG. 2A;

FIG. 3A is an enlarged, side, perspective view of a distal portion of another end effector assembly configured for use with the surgical instrument of the surgical system of FIG. 1;

FIG. 3B is a transverse, cross-sectional view of the end effector assembly of FIG. 3A;

FIG. 4A is an enlarged, side, perspective view of a distal portion of yet another end effector assembly configured for use with the surgical instrument of the surgical system of FIG. 1;

FIG. 4B is a transverse, cross-sectional view of the end effector assembly of FIG. 4A;

FIGS. 5A and 5B are flow diagrams illustrating a method of operating an instrument including both ultrasonic and electrosurgical functionality;

FIG. 6 is a flow diagram illustrating another method of operating an instrument including both ultrasonic and electrosurgical functionality; and

FIG. 7 is a transverse, cross-sectional view of a jaw configured for use with the surgical instrument of the surgical system of FIG. 1.

### DETAILED DESCRIPTION

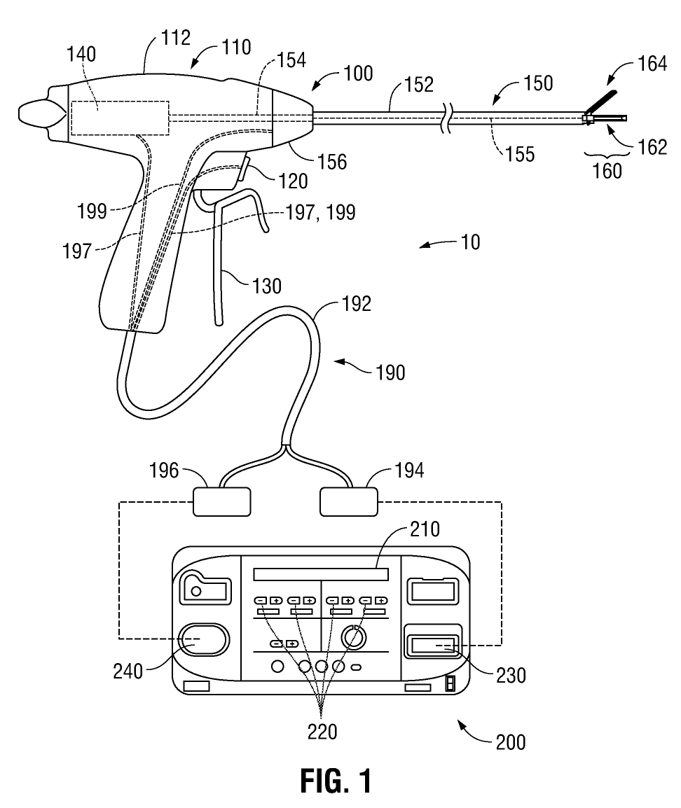

Referring to FIG. 1, a surgical system provided in accordance with aspects of the present disclosure is shown generally identified by reference numeral 10 including a surgical instrument 100 and a surgical generator 200. Surgical instrument 100 includes a handle assembly 110, an elongated assembly 150 extending distally from handle assembly 110, and a plug assembly 190 operably coupled with handle assembly 110 and extending therefrom for connection to surgical generator 200. As an alternative to handle assembly 110, surgical instrument 100 may include a robotic attachment housing for releasable engagement with a robotic arm of a robotic surgical system (not shown). Surgical generator 200 includes a display 210, a plurality user interface features 220, e.g., buttons, touch-screens, switches, etc., an ultrasonic plug port 230, and an electrosurgical plug port 240. Surgical generator 200 is configured to produce ultrasonic drive signals for output through ultrasonic plug port 230 to surgical instrument 100 to activate surgical instrument 100 in an ultrasonic mode of operation and to provide electrosurgical, e.g., RF bipolar energy, for output through electrosurgical plug port 240 to surgical instrument 100 to activate surgical instrument 100 in an electrosurgical mode of operation. It is also contemplated that a common port (not shown) configured to act as both ultrasonic plug port 230 and an electrosurgical plug port 240 may be utilized.

Handle assembly 110 includes a housing 112 defining a body portion and a fixed handle portion. Handle assembly 110 further includes an activation button 120 and a clamp trigger 130. The body portion of housing 112 is configured to support an ultrasonic transducer 140. Ultrasonic transducer 140 may be permanently engaged with the body portion of housing 112 or removable therefrom. Ultrasonic transducer 140 includes a piezoelectric stack or other suitable ultrasonic transducer components electrically coupled to surgical generator 200, e.g., via one or more of first electrical lead wires 197, to enable communication of ultrasonic drive signals to ultrasonic transducer 140 to drive ultrasonic transducer 140 to produce ultrasonic vibration energy that is transmitted along a waveguide 154 of elongated assembly 150 to blade 162 of end effector assembly 160 of elongated assembly 150, as detailed below. Activation button 120 is coupled to or between ultrasonic transducer 140 and/or surgical generator 200, e.g., via one or more of first electrical lead wires 197, to enable activation of ultrasonic transducer 140 in response to depression of activation button 120. In embodiments, activation button 120 may be an ON/OFF switch. In other embodiments, activation button 120 may include multiple actuated stages to enable activation from an OFF position to different actuated positions corresponding to different modes, e.g., a first actuated position corresponding to a first mode and a second actuated position corresponding to a second mode. In still other embodiments, separate activation buttons may be provided, e.g., a first actuation button for activating a first mode and a second activation button for activating a second mode.

Continuing with reference to FIG. 1, elongated assembly 150 of surgical instrument 100 includes an outer drive sleeve 152, an inner support sleeve (not shown) disposed within outer drive sleeve 152, a waveguide 154 extending through the inner support sleeve, a drive assembly (not shown), a rotation knob 156, and an end effector assembly 160 including a blade 162 and a jaw 164. The drive assembly operably couples a proximal portion of outer drive sleeve 152 to clamp trigger 130 of handle assembly 100, a distal portion of outer drive sleeve 152 is operably coupled to jaw 164, and a distal end of the inner support sleeve pivotably supports jaw 164. As such, clamp trigger 130 is selectively actuatable to thereby move outer drive sleeve 152 about the inner support sleeve to pivot jaw 164 relative to blade 162 of end effector assembly 160 from a spaced-apart position to an approximated position for clamping tissue between jaw 164 and blade 162. The drive assembly may provide a force-limiting feature whereby the clamping pressure applied to tissue clamped between jaw 164 and blade 162 is limited to a particular clamping pressure or particular clamping pressure range. Rotation knob 156 is rotatable in either direction to rotate elongated assembly 150 in either direction relative to handle assembly 110.

Waveguide 154, as noted above, extends from handle assembly 110 through the inner support sleeve. Waveguide 154 defines a body 155 and blade 162 extending from the distal end of body 155. Waveguide 154 (including blade 162) may be formed from titanium, a titanium alloy, or other suitable electrically-conductive material(s). Blade 162 serves as the blade of end effector assembly 160 and may be integrally formed with waveguide 154 or separately formed and subsequently attached (permanently or removably) to waveguide 154. Waveguide 154 further includes a proximal connector (not shown), e.g., a threaded male connector, configured for engagement, e.g., threaded engagement within a threaded female receiver, of ultrasonic transducer 140 such that ultrasonic motion produced by ultrasonic transducer 140 is transmitted along waveguide 154 to blade 162 for treating tissue clamped between blade 162 and jaw 164 or positioned adjacent to blade 162.

Plug assembly 190 of surgical instrument 100 includes a cable 192, an ultrasonic plug 194, and an electrosurgical plug 196. Ultrasonic plug 194 is configured for connection with ultrasonic plug port 230 of surgical generator 200 while electrosurgical plug 196 is configured for connection with electrosurgical plug port 240 of surgical generator 200. In embodiments where generator 200 includes a common port, plug assembly 190 may include a common plug (not shown) configured to act as both the ultrasonic plug 194 and the electrosurgical plug 196. Plural first electrical lead wires 197 electrically coupled to ultrasonic plug 194 extend through cable 192 and into handle assembly 110 for electrical connection to ultrasonic transducer 140 and/or activation button 120 to enable the selective supply of ultrasonic drive signals from surgical generator 200 to ultrasonic transducer 140 upon activation of activation button 120 in an ultrasonic mode of operation. In addition, plural second electrical lead wires 199 are electrically coupled to electrosurgical plug 196 and extend through cable 192 into handle assembly 110. Separate second electrical lead wires 199 are electrically connected to waveguide 154 and jaw 164 such that, as detailed below, bipolar electrosurgical energy may be conducted therebetween. One or more second electrical lead wires 199 is electrically coupled to activation button 120 to enable the selective supply of bipolar electrosurgical energy from surgical generator 200 to waveguide 154 and jaw 164 upon activation of activation button 120 in an electrosurgical mode of operation.

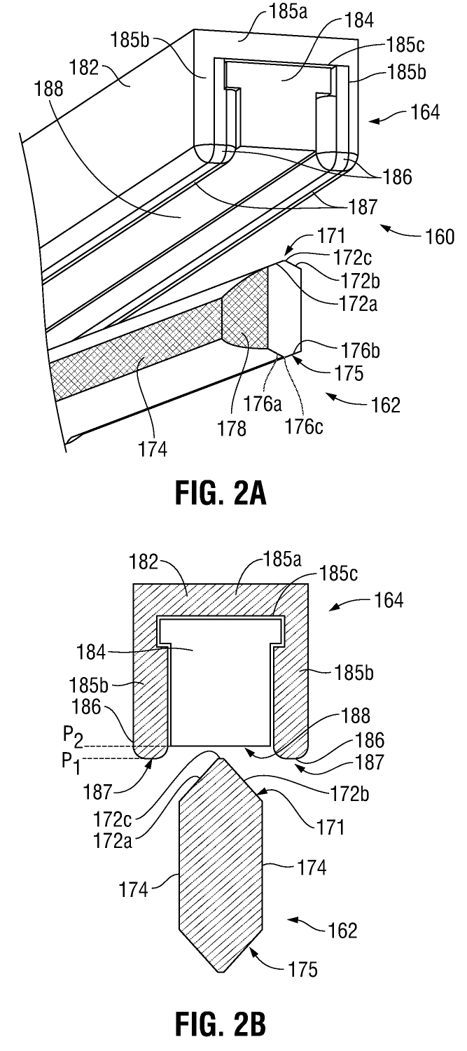

Referring to FIGS. 2A and 2B, blade 162 of end effector assembly 160 may define a linear configuration, may define a curved configuration, or may define any other suitable configuration, e.g., straight and/or curved surfaces, portions, and/or sections; one or more convex and/or concave surfaces, portions, and/or sections; etc. With respect to curved configurations, blade 162, more specifically, may be curved in any direction relative to jaw 164, for example, such that the distal tip of blade 162 is curved towards jaw 164, away from jaw 164, or laterally (in either direction) relative to jaw 164. Further, blade 162 may be formed to include multiple curves in similar directions, multiple curves in different directions within a single plane, and/or multiple curves in different directions in different planes. In addition, although one configuration of blade 162 is described and illustrated herein, it is contemplated that blade 162 may additionally or alternatively be formed to include any suitable additional or alternate features, e.g., a tapered configuration, various different cross-sectional configurations along its length, cut-outs, indents, edges, protrusions, straight surfaces, curved surfaces, angled surfaces, wide edges, narrow edges, and/or other features.

In embodiments, blade 162 defines a generally convex first tissue-contacting surface 171, e.g., the surface that opposes jaw 164. Generally convex first tissue-contacting surface 171 may defined by a pair of surfaces 172a, 172b (flat or arcuate surfaces) that converge at an apex 172c, or may be formed by a single arcuate surface defining an apex 172c. Blade 162 may further define substantially flat lateral surfaces 174 (excluding any curvature due to the curvature of blade 162 itself) on either side of first tissue-contacting surface 171, and a second tissue-contacting surface 175 opposite first tissue-contacting surface 171 and similarly configured relative thereto, e.g., with surfaces 176a, 176b converging at an apex 176c, although other configurations are also contemplated.

Waveguide 154, or at least the portion of waveguide 154 proximally adjacent blade 162, may define a cylindrical-shaped configuration. Plural tapered surfaces (not shown) may interconnect the cylindrically-shaped waveguide 154 with the polygonal (or rounded-edge polygonal) configuration of blade 162 to define smooth transitions between the body of waveguide 154 and blade 162. Additionally or alternatively, inwardly tapered surfaces 178 may extend from lateral surfaces 174 at the distal end of blade 162 such that the distal end of blade 162 defines a narrowed configuration as compared to the body of blade 162.

First tissue-contacting surface 171 is configured to contact tissue clamped between blade 162 and jaw 164 for treating clamped tissue, e.g., sealing and/or cutting clamped tissue, while second tissue-contacting surface 175 may be utilized for, e.g., tissue dissection, back scoring, etc. More specifically, in an ultrasonic mode of operation, ultrasonic energy transmitted along waveguide 154 to blade 162 may be utilized to treat tissue clamped between first tissue-contacting surface 171 of blade 162 and jaw 164 and/or to treat tissue in contact with second tissue-contacting surface 175. In an electrosurgical mode of operation, bipolar electrosurgical energy may be conducted from first tissue-contacting surface 171 of blade 162 to jaw 164 through tissue clamped therebetween to treat clamped tissue. Other suitable configurations for bipolar electrosurgical energy-based tissue treatment are also contemplated. Monopolar electrosurgical activation, e.g., wherein blade 162 and jaw 164 are energized to the same potential (or one is isolated), for electrosurgical dissection and/or spot coagulation utilizing second tissue-contacting surface 175, used together with a remote return pad or local return device, is also contemplated as are any other additional or alternative suitable arrangements. It is noted that the ultrasonic and electrosurgical modes need not be mutually exclusive but, rather, can operate simultaneously, overlapping, consecutively, alternatingly, or in any other suitable manner.

As illustrated in FIG. 2A, lateral surfaces 174 and, in embodiments, tapered surfaces 178 and/or the proximal tapered surfaces (not shown), may be coated with an electrically-insulative material such that, in the electrosurgical mode of operation, current is directed from first tissue-contacting surface 171 of blade 162 to jaw 164 rather than from lateral surfaces 174 (or tapered surfaces 178 or the proximal tapered surfaces (not shown)). Suitable electrically-insulative coatings and/or methods of applying coatings include but are not limited to Teflon®, polyphenylene oxide (PPO), deposited liquid ceramic insulative coatings; thermally sprayed coatings, e.g., thermally sprayed ceramic; Plasma Electrolytic Oxidation (PEO) coatings; anodization coatings; sputtered coatings, e.g., silica; ElectroBond® coating available from Surface Solutions Group of Chicago, IL, USA; or other suitable coatings and/or methods of applying coatings. Structural body 182 may additionally or alternatively be selectively coated with electrical insulation such that, in the electrosurgical mode of operation, current is directed between first tissue-contacting surface 171 of blade 162 and tissue-contacting surfaces 187 of jaw 164 rather than between surfaces 185a, 185b and the exterior surfaces of jaw 182.

Referring again to FIGS. 2A and 2B, jaw 164 of end effector assembly 160 includes a more-rigid structural body 182 and a more-compliant jaw liner 184. Structural body 182 includes a pair of proximal flanges (not shown) that are pivotably coupled to the inner support sleeve of surgical instrument 100 (FIG. 1) and operably associated with outer drive sleeve 152 (FIG. 1), e.g., via receipt within apertures defined within outer drive sleeve 152 (FIG. 1), such that sliding of outer drive sleeve 152 (FIG. 1) about the inner support sleeve pivots jaw 164 relative to blade 162 from a spaced-apart position to an approximated position to clamp tissue between jaw liner 184 of jaw 164 and blade 162. Structural body 182 further includes an elongated distal portion defining a generally U-shaped configuration including a backspan 185a and a pair of spaced-apart uprights 185b extending from backspan 185a in generally perpendicular orientation relative to backspan 185a and generally parallel orientation relative to one another. “Generally,” “about,” “substantially,” and like terms are utilized herein to take into account tolerances such as, for example, material, manufacturing, environmental, and/or use tolerances, and may include up to 10% variation.

Backspan 185a and uprights 185b cooperate to define a cavity 185c therein. Cavity 185c defines an elongated, generally T-shaped configuration for slidable receipt and retention of jaw liner 184 therein, although other suitable configurations for receiving and retaining jaw liner 184 are also contemplated.

Structural body 182 is adapted to connect to a source of electrosurgical energy, e.g., via one of lead wire 199 (FIG. 1), and, in the electrosurgical mode of operation, is charged to a different potential as compared to blade 162 to enable the conduction of bipolar electrosurgical (e.g., RF) energy therebetween, through tissue disposed therebetween, to treat the tissue. More specifically, electrosurgical energy is configured to flow between first tissue-contacting surface 171 of blade 162 and free ends 186 of uprights 185b of structural body 182 and through tissue disposed therebetween to complete the electrosurgical energy circuit (in conjunction with lead wires 199 (FIG. 1)). Free ends 186 of uprights 185b of structural body 182 may define rounded edges to inhibit current concentrations and, in embodiments, define tissue-contacting surfaces 187 that extend transversely, in generally perpendicular orientation relative to backspan 185a and generally parallel orientation relative to uprights 185b. Tissue contacting surfaces 187 cooperate to define a transverse plane “P1.” Uprights 185b are spaced a greater distance than the width of blade 162 such that, in the approximated position of jaw 164, neither tissue contacting surfaces 187 nor any other portions of structural body 182 contact blade 162.

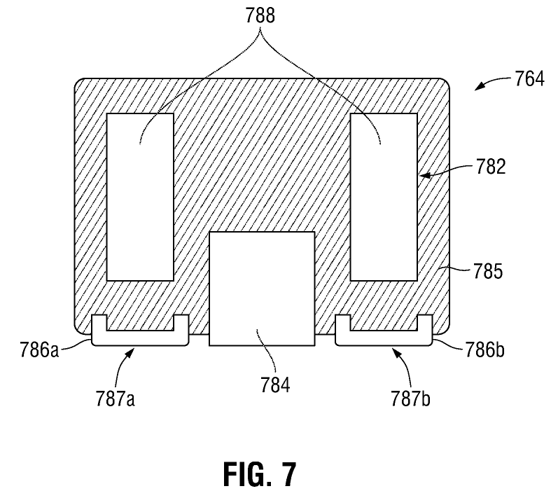

In embodiments, the structural body may alternatively define a relatively smaller footprint and be embedded in an insulative material, e.g., an overmolded plastic. In such embodiments, electrically-conductive plates may be disposed on or captured by the overmolded plastic to function as the free ends of the uprights and enable electrical conduction of energy. More specifically, with momentary reference to FIG. 7, another jaw 764 configured for use with end effector assembly 160 (FIGS. 2A and 2B) or other suitable end effector assembly includes a more-rigid structural body 782, a more-compliant jaw liner 784, an insulative housing 785, and first and second electrically-conductive plates 786a, 786b defining respective tissue-contacting surfaces 787a, 787b.

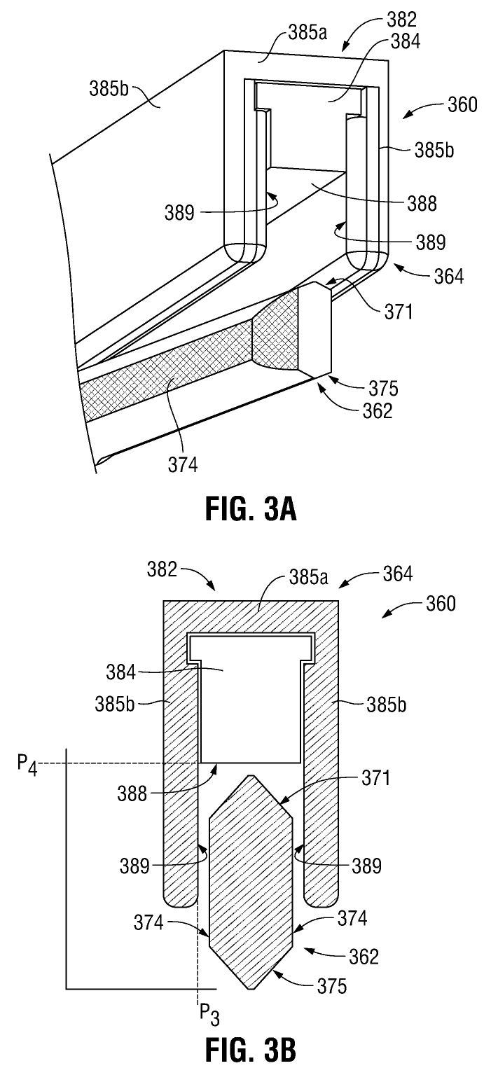

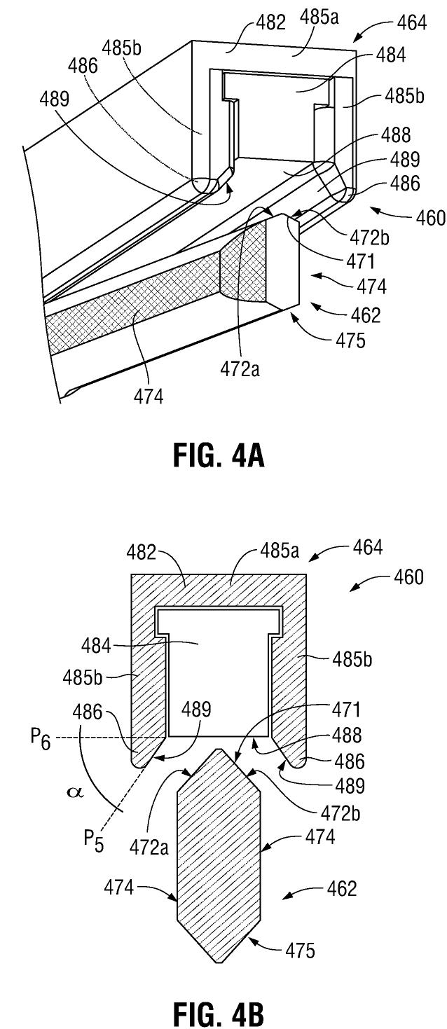

Structural body 782 includes a pair of proximal flanges (not shown) that are pivotably coupled to the inner support sleeve of a surgical instrument, e.g., surgical instrument 100 (FIG. 1) and operably associated with a drive feature thereof, e.g., outer drive sleeve 152 (FIG. 1) such that actuation of the drive feature pivots jaw 764 relative to a blade of the end effector, e.g., blade 162 (FIGS. 2A and 2B), from a spaced-apart position to an approximated position to clamp tissue between jaw liner 784 of jaw 764 and the blade. Structural body 782 further includes an elongated distal portion defining a pair of spaced-apart upright supports 788 which may be separate from one another along the lengths thereof or joined via a backspan (not shown) along at least portions of the lengths thereof). Insulative housing 785 is formed via overmolding, e.g., with one or multiple-shot overmolds, or is otherwise configured, and serves to capture and retain structural body 782, jaw liner 784, and first and second electrically-conductive plates 786a, 786b in position relative to one another. Insulative housing 785 and/or electrically-conductive plates 786a, 786b are not limited to the configuration illustrated in FIG. 7 but, rather, may define any suitable configuration to achieve a desired height, angle, etc. of electrically-conductive plates 786a, 786b relative to one another and/or the tissue contacting surface of jaw liner 784, e.g., to achieve the configuration shown in FIGS. 3A and 3B or FIGS. 4A and 4B.

Referring back to FIGS. 2A and 2B, jaw liner 184 is shaped complementary to cavity 185c, e.g., defining a T-shaped configuration, for receipt and retention therein and is fabricated from a compliant material such as, for example, polytetrafluoroethylene (PTFE), such that blade 162 is permitted to vibrate while in contact with jaw liner 184 without damaging components of ultrasonic surgical instrument 10 (FIG. 1) and without compromising the hold on tissue clamped between jaw 164 and blade 162. Other suitable materials are also contemplated. Jaw liner 184 includes a tissue contacting surface 188 that is substantially planar (not withstanding gripping teeth and/or indentations formed therein) and defines a transverse plane “P2” that is substantially parallel with transverse plane “P1.” Free ends 186 of uprights 185b of structural body 182 extend beyond transverse plane “P2” towards blade 162 such that transverse plane “P1” is spaced-apart from transverse plane “P2” and closer to blade 162. In other embodiments, planes “P1,” “P2” may be substantially coplanar.

As a result of the above-detailed configuration of end effector assembly 160, with tissue clamped between blade 162 and jaw 164 in the approximated position of jaw 164, tissue is held by and in contact with tissue contacting surface 188 of jaw liner and tissue-contacting surfaces 187 of uprights 185b of structural body 182 on the jaw side of tissue, and first tissue-contacting surface 171 on the blade side of tissue. Upon activation in the electrosurgical mode, electrosurgical energy is substantially conducted from first tissue-contacting surface 171, through the clamped tissue, to tissue-contacting surfaces 187 of uprights 185b to, for example, seal the clamped tissue. It is noted that some of the electrosurgical energy may find other current paths, e.g., from first tissue-contacting surface 171 to different portions of uprights 185b. An electrical insulating layer may additionally or alternatively be applied to portions of structural body 182 and blade 162 to inhibit electrosurgical energy from finding alternate current paths. Upon activation in the ultrasonic mode, ultrasonic energy is transmitted from first tissue-contacting surface 171 to tissue clamped between first tissue-contacting surface 171 and tissue contacting surface 188 of jaw liner 184, e.g., to cut tissue. In this manner, the electrosurgical mode may be activated and, sequentially thereafter, overlapping thereafter, or concurrently therewith, the ultrasonic mode may be activated to seal tissue and cut tissue between sealed portions thereof. Other configurations of activation, such as those detailed below, are also contemplated, for example, to achieve a desired tissue effect or effects, e.g., sealing, cutting, etc.

Turning to FIGS. 3A and 3B, another end effector assembly 360 configured for use with surgical instrument 100 (FIG. 1) is shown including a blade 362 and a jaw 364. End effector assembly 360 is similar to end effector assembly 160 (FIGS. 1-2B) and, thus, only differences therebetween are described in detail below while similarities are summarily described or omitted entirely.

Blade 362 of end effector assembly 360 includes a first tissue-contacting surface 371 that generally opposes jaw 364, lateral surfaces 374 (which may be coated with an electrically-insulative material), and a second tissue-contacting surface 375 opposite first tissue-contacting surface 371.

Jaw 364 of end effector assembly 360 includes a more-rigid structural body 382 and a more-compliant jaw liner 384. Structural body 382 further includes an elongated distal portion defining a generally U-shaped configuration including a backspan 385a and a pair of spaced-apart uprights 385b extending from backspan 385a in generally perpendicular orientation relative to backspan 385a and generally parallel orientation relative to one another. Uprights 385b define opposed, inwardly-facing tissue contacting surfaces 389 that are substantially parallel with one another and uprights 385b. Inwardly-facing tissue contacting surfaces 389 define planes “P3.”

Structural body 382 is adapted to connect to a source of electrosurgical energy, e.g., via one of lead wire 199 (FIG. 1), and, in the electrosurgical mode of operation, is charged to a different potential as compared to blade 362 to enable the conduction of bipolar electrosurgical (e.g., RF) energy therebetween to treat tissue. More specifically, electrosurgical energy is configured to flow from first tissue-contacting surface 371 of blade 362, through tissue, to inwardly-facing tissue contacting surfaces 389 of uprights 385b of structural body 382 for return to the electrosurgical energy source via the lead wire 199 (FIG. 1).

Jaw liner 384 is configured for receipt and retention within structural body 382. Jaw liner 384 includes a tissue contacting surface 388 defining a transverse plane “P4” that is substantially perpendicular to planes “P3.” Jaw liner 384 defines a reduced height as compared to uprights 385b such that tissue contacting surface 388 is recessed within structural body 382 and such that portions of inwardly-facing tissue contacting surfaces 389 of uprights 385b are exposed. Further, as a result of this configuration, in the approximated position of jaw 364, blade 362 is at least partially received within structural body 382, e.g., wherein lateral surfaces 374 are at least partially overlapping with (and disposed in parallel orientation relative to) inwardly-facing tissue contacting surfaces 389 of uprights 385b.

With tissue clamped between blade 362 and jaw 364 in the approximated position of jaw 364, tissue is held by and in contact with inwardly-facing tissue contacting surfaces 389 of uprights 385b of structural body 382 and tissue contacting surface 388 of jaw liner 384 on the jaw side of tissue, and first tissue-contacting surface 371 of blade 362 on the blade side of tissue. Upon activation in the electrosurgical mode, electrosurgical energy is substantially conducted from first tissue-contacting surface 371, through the clamped tissue, to inwardly-facing tissue contacting surfaces 389 to, for example, seal the clamped tissue. It is noted that some of the electrosurgical energy may find other current paths, e.g., from first tissue-contacting surface 371 to different portions of uprights 385b. Structural body 382 may additionally or alternatively be selectively coated with electrical insulation similarly as detailed above with respect to previous embodiments, in order to inhibit alternate current paths. Upon activation in the ultrasonic mode, ultrasonic energy is transmitted from first tissue-contacting surface 371 to tissue clamped between first tissue-contacting surface 371 and tissue contacting surface 388 of jaw liner 384, e.g., to cut tissue.

With reference to FIGS. 4A and 4B, another end effector assembly 460 configured for use with surgical instrument 100 (FIG. 1) is shown including a blade 462 and a jaw 464. End effector assembly 460 is similar to end effector assembly 160 (FIGS. 1-2B) and, thus, only differences therebetween are described in detail below while similarities are summarily described or omitted entirely.

Blade 462 of end effector assembly 460 includes a first tissue-contacting surface 471 that generally opposes jaw 464, lateral surfaces 474 (which may be coated with an electrically-insulative material), and a second tissue-contacting surface 475 opposite first tissue-contacting surface 471.

Jaw 464 of end effector assembly 460 includes a more-rigid structural body 482 and a more-compliant jaw liner 484. Structural body 482 further includes an elongated distal portion defining a generally U-shaped configuration including a backspan 485a and a pair of spaced-apart uprights 485b extending from backspan 485a in generally perpendicular orientation relative to backspan 185a and generally parallel orientation relative to one another. Uprights 485b include beveled free ends 486 defining inwardly-angled tissue-contacting surfaces 489. Each inwardly-angled tissue-contacting surface 489 defines a plane (only plane “P5” of one of the inwardly-angled tissue-contacting surfaces 489 is identified in FIG. 4B). In embodiments, inwardly-angled tissue-contacting surfaces 489 of uprights 485b are disposed in substantially parallel orientation relative to respective surfaces 472a, 472b that define first tissue-contacting surface 471 in the approximated position of jaw 464.

Structural body 482 is adapted to connect to a source of electrosurgical energy, e.g., via one of lead wire 199 (FIG. 1), and, in the electrosurgical mode of operation, is charged to a different potential as compared to blade 462 to enable the conduction of bipolar electrosurgical (e.g., RF) energy therebetween to treat tissue. More specifically, electrosurgical energy is configured to flow from first tissue-contacting surface 471 of blade 462, through tissue, to inwardly-angled tissue-contacting surfaces 489 of uprights 485b of structural body 482 for return to the electrosurgical energy source via the lead wire 199 (FIG. 1). Electrically-insulative coatings may additionally or alternatively be applied to surfaces of structural body 482 to inhibit current flow through tissue to/from surfaces other than the tissue-contacting surfaces 489.

Jaw liner 484 is configured for receipt and retention within structural body 482. Jaw liner 484 includes a tissue contacting surface 488 defining a transverse plane “P6” that is disposed at an acute angle “a” relative to the plane “P5” of each inwardly-angled tissue-contacting surface 489.

With tissue clamped between blade 462 and jaw 464 in the approximated position of jaw 464, tissue is held by and in contact with inwardly-angled tissue-contacting surfaces 489 of uprights 485b of structural body 482 and tissue contacting surface 488 of jaw liner 484 on the jaw side of tissue, and first tissue-contacting surface 471 of blade 462 on the blade side of tissue. Upon activation in the electrosurgical mode, electrosurgical energy is substantially conducted from first tissue-contacting surface 471 (and, more specifically, from surfaces 472a, 472b thereof), through the clamped tissue, to inwardly-angled tissue contacting surfaces 489 to, for example, seal the clamped tissue. Notably, surfaces 472a, 472b and respective surfaces 489 may define parallel electrode surfaces in some embodiments. Upon activation in the ultrasonic mode, ultrasonic energy is transmitted from first tissue-contacting surface 471 to tissue clamped between first tissue-contacting surface 471 and tissue contacting surface 488 of jaw liner 484, e.g., to cut tissue.

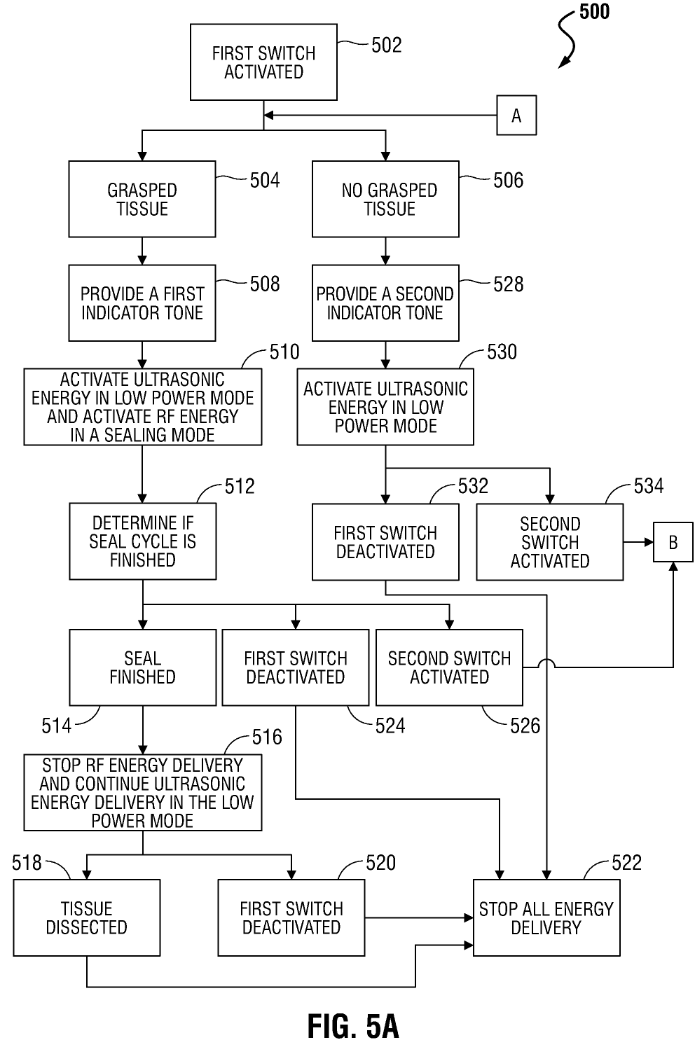

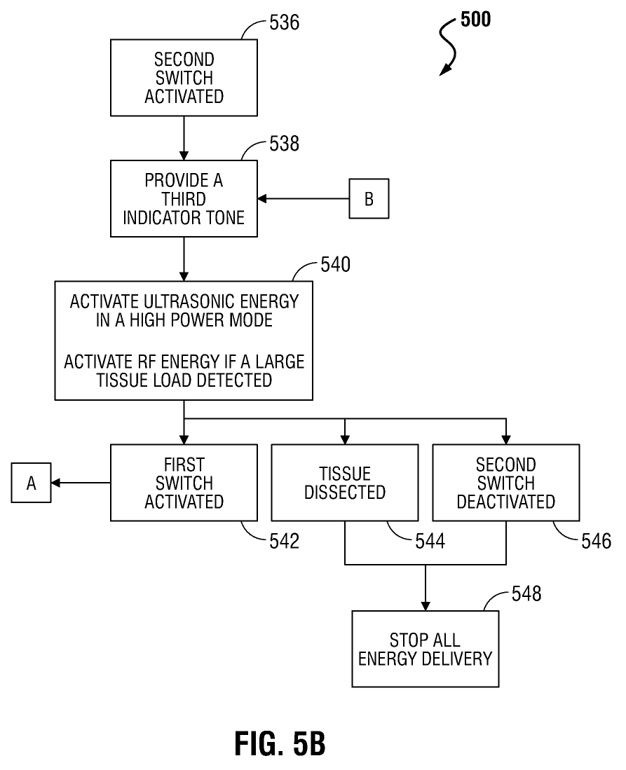

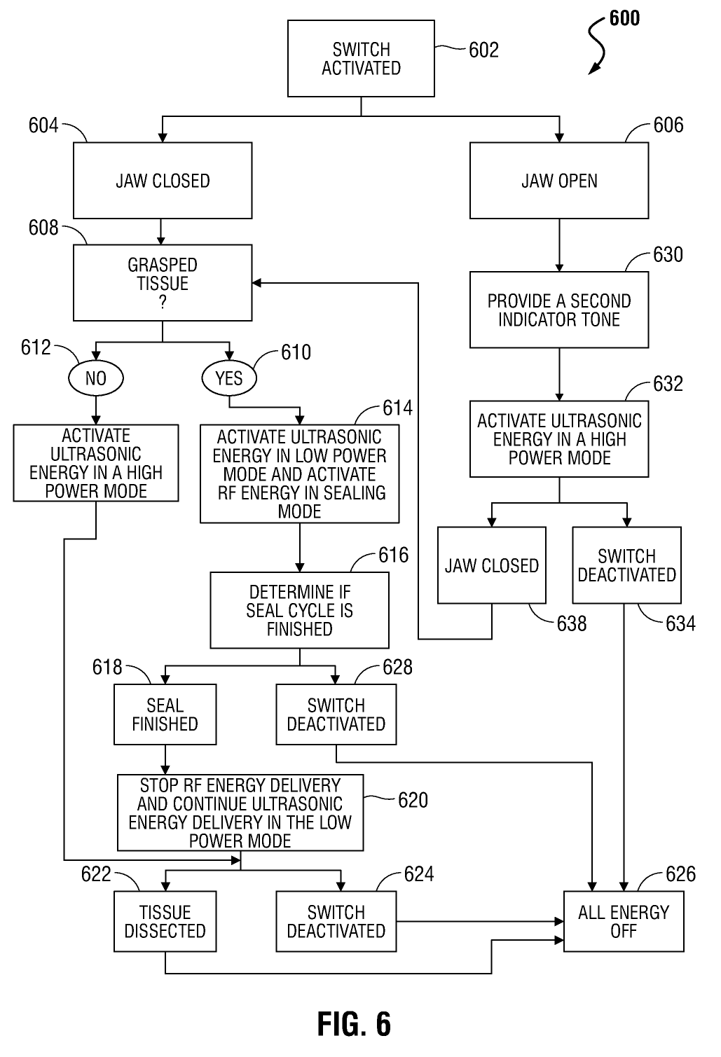

Referring to FIGS. 5A-6, methods of selectively activating electrosurgical and/or ultrasonic modes of operation of a combination electrosurgical and ultrasonic surgical instrument, e.g., surgical instrument 100 (FIG. 1), to treat, e.g., seal and/or cut, tissue are illustrated as methods 500 (FIGS. 5A-5B) and 600 (FIG. 6). More specifically, method 500 (FIGS. 5A and 5B) is for use with a surgical instrument including two activation switches (whether incorporated into a single activation button or separate activation buttons) while method 600 (FIG. 6) is for use with a surgical instrument including a single activation switch. With respect to method 500, it is understood that, whether by physical and/or electrical (hardware and/or software) features, the two activation switches are inhibited from simultaneous effective activation.

Turning to FIG. 5A, with respect to method 500, it is initially determined whether the first switch is activated at 502 or whether the second switch is activated at 236 (FIG. 5B). If it determined that the first switch is activated at 502, it is then determined whether tissue is grasped at 504, e.g., between jaw 164 and blade 162 (FIGS. 1-2B), or whether there is no tissue grasped at 506. Determining whether tissue is grasped or not at 504, 506, respectively, may be accomplished via sensing impedance between the electrosurgical electrodes (jaw 164 and blade 162 (FIGS. 1-2B), determining whether jaw 164 (FIGS. 1-2B)) is disposed in the approximated position, or in any other suitable manner.

If it is determined that tissue is grasped at 504, a first indicator tone is provided at 508; on the other hand, if it is determined that tissue is not grasped at 506, a second indicator tone is provided at 528. The first and second indicator tones alert the user that the first switch has been activated and it has been determined that tissue is grasped or not grasped, respectively. Any suitable differentiable tones or other audio may be utilized; additionally or alternatively, other suitable feedback may be provided, e.g., visual, tactile, combinations thereof, etc.

Where it is determined that tissue is grasped at 504 and, after the corresponding tone at 508, the surgical instrument is activated in both the ultrasonic mode of operation and in the electrosurgical mode of operation at 510. In some embodiments, such as where the ultrasonic mode of operation includes multiple power levels, e.g., a LOW power lever and a HIGH power level (although other levels are also contemplated), the ultrasonic mode of operation may be activated on LOW at 510. As an alternative to multiple power levels, two different ultrasonic sub-modes of operation may be utilized (wherein a first ultrasonic sub-mode replaces the HIGH power lever and a second ultrasonic sub-mode replaces the LOW power level). The ultrasonic sub-modes may include different feedback-based algorithms, different set points, etc. Thus, the ultrasonic energy may be activated in any suitable (pre-set or variable) sub-mode and/or at any suitable (pre-set or variable) power level when the surgical instrument is activated in both the ultrasonic mode of operation and in the electrosurgical mode of operation at 510.

While the surgical instrument is activated in both the ultrasonic and electrosurgical modes of operation at 510 it is determined, e.g., continuously, periodically, etc., whether a seal cycle has been completed at 512. This may be determined by monitoring electrosurgical impedance, power, combinations thereof, or in any other suitable manner. If it is determined that the seal cycle is completed and, thus, the grasped tissue is sealed, as noted at 514, the method proceeds to 516 where the electrosurgical mode is deactivated while the ultrasonic mode is continued, e.g., on LOW, to dissect tissue. A tone may also be provided to indicate seal and/or dissection completion. Once tissue is dissected, as indicated at 518, the ultrasonic mode is deactivated (so no energy is being delivered), as indicated at 522. Tissue dissection may be determined by monitoring electrosurgical impedance, power, combinations thereof, or in any other suitable manner. If the first switch is deactivated at any point in the above, e.g., at 520 or at 524, all energy delivery is ceased, as indicated at 522. If the second switch is activated at 526, the method proceeds to 538 (FIG. 5B).

Returning to 502, where it is determined that the first switch is activated at 502, that tissue is not grasped at 506, and after the corresponding tone at 528, the surgical instrument is activated in the ultrasonic mode of operation, e.g., on LOW, as indicated at 530, and maintained until either the first switch is deactivated at 532 or the second switch is activated at 534. In embodiments where more than two settings are provided, the LOW setting may be utilized at 510, an intermediate setting may be used at 530, and a HIGH setting may be used at 540, for example. Where the first switch is deactivated at 532, all energy delivery is ceased, as indicated at 522. The above activation where tissue is not grasped allows for ultrasonic dissection, e.g., using either the upper or lower tissue-contacting surface of the ultrasonic blade of the surgical instrument.

With reference to FIG. 5B, where it is determined that second switch is activated, e.g., initially at 536 or as previously noted at 526 or 534 (see FIG. 5A), a third tone is provided at 538 to alert the user that the second switch has been activated. Any suitable tone differentiable from the first and second tones utilized; additionally or alternatively, other suitable feedback may be provided, e.g., visual, tactile, combinations thereof, etc.

After providing the third tone at 538, the surgical instrument is activated in the ultrasonic mode of operation at 540. In embodiments where the ultrasonic mode of operation includes multiple power levers, the ultrasonic mode of operation may be activated on HIGH at 540. Further, where a load above a threshold load, large-diameter tissue is detected, e.g., clamped between the jaw and blade, and/or in other situations, the activation at 540 may further include activating the electrosurgical mode of operation simultaneously with the ultrasonic mode of operation.

The activation at 540 continues until the first switch is activated at 542, it is determined that tissue has been dissected at 544, or the second switch is deactivated at 546. Where it is determined that tissue has been dissected at 544 (e.g., by monitoring electrosurgical impedance, power, combinations thereof, or in any other suitable manner) or the second switch is deactivated at 546, energy delivery is ceased at 548. On the other hand, where the first switch is activated at 542, the method returns to 502 (FIG. 5A) and proceeds as detailed above.

Turning to FIG. 6, with respect to method 600, it is initially determined whether the switch is activated at 602. If it determined that the switch is activated at 602, it is then determined whether the jaw is closed at 604 or whether the jaw is open at 606. This may be determined by determining whether jaw 164 (FIGS. 1-2B) is disposed in the spaced-apart position or the approximated position, or in any other suitable manner.

Where the jaw is determined to be closed at 604, it is then determined at 608 whether tissue is grasped, e.g., between jaw 164 and blade 162 (FIGS. 1-2B), or whether there is no tissue grasped. This may be accomplished via sensing impedance between the electrosurgical electrodes (jaw 164 and blade 162 (FIGS. 1-2B), or in any other suitable manner.

If it is determined that tissue is grasped, “YES” at 610, the surgical instrument is activated in both the ultrasonic mode of operation and in the electrosurgical mode of operation at 614. In embodiments where the ultrasonic mode of operation includes multiple power levers, e.g., a LOW power lever and a HIGH power lever, the ultrasonic mode of operation may be activated on LOW at 614.

While the surgical instrument is activated in both the ultrasonic and electrosurgical modes of operation at 614, it is determined, e.g., continuously, periodically, etc., whether a seal cycle has been completed at 616. This may be determined by monitoring electrosurgical impedance, power, combinations thereof, or in any other suitable manner. If it is determined that the seal cycle is completed and, thus, the grasped tissue is sealed, as indicated at 618, the method proceeds to 620 where the electrosurgical mode is deactivated while the ultrasonic mode is continued, e.g., on LOW (or at any other suitable power level and/or in any other suitable sub-mode), to dissect tissue. A tone may also be provided to indicate seal and/or dissection completion. Once tissue is dissected, as indicated at 622, the ultrasonic mode is deactivated (so no energy is being delivered), as indicated at 626. Tissue dissection may be determined by monitoring electrosurgical impedance, power, combinations thereof, or in any other suitable manner. If the switch is deactivated at any point in the above, e.g., at 624 or at 628, all energy delivery is ceased, as indicated at 626.

Returning to 602, where it is determined that the switch is activated at 602 and that the jaw is open at 606, a tone is provided at 630 to alert the user that the switch has been activated and that the jaw is open.

After providing the tone at 630, the surgical instrument is activated in the ultrasonic mode of operation at 632. In embodiments where the ultrasonic mode of operation includes multiple power levers, the ultrasonic mode of operation may be activated on HIGH at 632. Further, where a load above a threshold load, large-diameter tissue is detected, and/or in other situations, the activation at 632 may further include activating the electrosurgical mode of operation simultaneously with the ultrasonic mode of operation.

The activation at 632 continues until it is determined that the jaw is closed at 636 or the switch is deactivated at 634. Where it is determined that the jaw has been closed, the method returns to 608 and proceeds therefrom as detailed above. Where it is determined that the switch is deactivated at 634, energy delivery is ceased at 636.

Referring back to FIG. 1, as opposed to a handle assembly 100 for handheld, manual manipulation and operation, the various embodiments disclosed herein may also be configured to work with robotic surgical systems and what is commonly referred to as “Telesurgery.” Such systems employ various robotic elements to assist the surgeon and allow remote operation (or partial remote operation) of surgical instrumentation. Various robotic arms, gears, cams, pulleys, electric and mechanical motors, etc. may be employed for this purpose and may be designed with a robotic surgical system to assist the surgeon during the course of an operation or treatment. Such robotic systems may include remotely steerable systems, automatically flexible surgical systems, remotely flexible surgical systems, remotely articulating surgical systems, wireless surgical systems, modular or selectively configurable remotely operated surgical systems, etc.

The robotic surgical systems may be employed with one or more consoles that are next to the operating theater or located in a remote location. In this instance, one team of surgeons or nurses may prep the patient for surgery and configure the robotic surgical system with one or more of the instruments disclosed herein while another surgeon (or group of surgeons) remotely control the instruments via the robotic surgical system. As can be appreciated, a highly skilled surgeon may perform multiple operations in multiple locations without leaving his/her remote console which can be both economically advantageous and a benefit to the patient or a series of patients.

The robotic arms of the surgical system are typically coupled to a pair of master handles by a controller. The handles can be moved by the surgeon to produce a corresponding movement of the working ends of any type of surgical instrument (e.g., end effectors, graspers, knifes, scissors, etc.) which may complement the use of one or more of the embodiments described herein. The movement of the master handles may be scaled so that the working ends have a corresponding movement that is different, smaller or larger, than the movement performed by the operating hands of the surgeon. The scale factor or gearing ratio may be adjustable so that the operator can control the resolution of the working ends of the surgical instrument(s).

The master handles may include various sensors to provide feedback to the surgeon relating to various tissue parameters or conditions, e.g., tissue resistance due to manipulation, cutting or otherwise treating, pressure by the instrument onto the tissue, tissue temperature, tissue impedance, etc. As can be appreciated, such sensors provide the surgeon with enhanced tactile feedback simulating actual operating conditions. The master handles may also include a variety of different actuators for delicate tissue manipulation or treatment further enhancing the surgeon’s ability to mimic actual operating conditions.

While several embodiments of the disclosure have been detailed above and are shown in the drawings, it is not intended that the disclosure be limited thereto, as it is intended that the disclosure be as broad in scope as the art will allow and that the specification be read likewise. Therefore, the above description and accompanying drawings should not be construed as limiting, but merely as exemplifications of particular embodiments. Those skilled in the art will envision other modifications within the scope and spirit of the claims appended hereto.

What is claimed is:

1. A surgical instrument, comprising:
a housing;
an ultrasonic transducer supported by the housing; and
an elongated assembly extending distally from the housing, the elongated assembly including:
a waveguide formed from an electrically-conductive material and adapted to connect to a source of electrosurgical energy at a first potential, the waveguide operably coupled to the ultrasonic transducer and including a blade at a distal end portion thereof, the blade defining an upper tissue-contacting surface, a lower tissue-contacting surface, and first and second lateral surfaces disposed between the upper and lower tissue-contacting surfaces, wherein the first and second lateral surfaces are coated with a material; and
a jaw pivotable relative to the blade between a spaced-apart position and an approximated position for grasping tissue between the blade and the jaw, the jaw including:
a structural base formed from an electrically-conductive material and adapted to connect to a source of electrosurgical energy at a second potential different from the first potential, wherein the structural base includes a backspan and first and second uprights extending from the backspan in spaced-apart relation to define a cavity therebetween; and
a jaw liner supported within the cavity of the structural base, the jaw liner positioned to oppose the upper tissue-contacting surface of the blade in the approximated position with the first and second uprights disposed on either side of the blade,
wherein, in an ultrasonic mode of operation, ultrasonic energy is produced by the ultrasonic transducer and transmitted along the waveguide to the blade for treating tissue in contact with the blade, and
wherein, in an electrosurgical mode of operation, electrosurgical energy is conducted between the blade and the first and second uprights to treat tissue disposed therebetween.

2. The surgical instrument according to claim 1, wherein each of the first and second uprights defines a tissue-contacting surface at the free end thereof.

3. The surgical instrument according to claim 2, wherein the tissue-contacting surfaces of the first and second uprights are disposed in substantially parallel orientation relative to a tissue-contacting surface of the jaw liner.

4. The surgical instrument according to claim 2, wherein the tissue-contacting surfaces of the first and second uprights are angled inwardly towards one another and at an angle relative to a tissue-contacting surface of the jaw liner.

5. The surgical instrument according to claim 1, wherein each of the first and second uprights defines an inwardly-facing tissue-contacting surface.

6. The surgical instrument according to claim 5, wherein the tissue-contacting surfaces of the first and second uprights are disposed in substantially perpendicular orientation relative to a tissue-contacting surface of the jaw liner.

7. The surgical instrument according to claim 6, wherein, in the approximated position, the first and second lateral surfaces of the blade at least partially overlap with the tissue-contacting surfaces of the first and second uprights.

8. The surgical instrument according to claim 1, wherein the lateral surfaces of the blade are disposed in substantially parallel orientation relative to one another.

9. The surgical instrument according to claim 8, wherein the lateral surface of the blade are disposed in substantially parallel orientation with tissue-contacting surfaces of the first and second uprights.

10. The surgical instrument according to claim 1, wherein the upper tissue-contacting surface of the blade includes first and second surfaces meeting at an apex.

11. The surgical instrument according to claim 10, wherein the first and second surfaces of the upper-tissue contacting surface of the blade are substantially parallel with respective angled tissue-contacting surfaces of the first and second uprights.

12. The surgical instrument according to claim 1, wherein the material is an electrically-insulative material selected from Teflon or polyphenylene oxide (PPO).

13. The surgical instrument according to claim 1, wherein inwardly-tapered surfaces extend from the first and second lateral surfaces of the blade at a distal end portion of the blade.

14. The surgical instrument according to claim 13, wherein the inwardly-tapered surfaces are coated with an electrically-insulative material.

15. The surgical instrument according to claim 1, wherein the waveguide includes a body and the blade extending distally from the body.

16. The surgical instrument according to claim 15, wherein the body is generally cylindrical and wherein tapered surfaces are defined between the generally cylindrical body and the first and second tissue-contacting surfaces of the blade.

17. The surgical instrument according to claim 15, wherein the body is generally cylindrical and wherein tapered surfaces are defined between the generally cylindrical body and the first and second lateral surfaces of the blade.

18. The surgical instrument according to claim 1, further comprising a plug assembly including an ultrasonic plug adapted to connect to an ultrasonic plug port of a surgical generator and an electrosurgical plug adapted to connected to an electrosurgical plug port of a surgical generator.

19. The surgical instrument according to claim 1, further comprising at least one activation switch supported by the housing, the at least one activation switch configured to selectively initiate at least one of the ultrasonic mode of operation or the electrosurgical mode of operation.

20. The surgical instrument according to claim 1, wherein the jaw liner is formed from a compliant material.
